(19) United States
(12) Patent Application Publication
(10) Pub. No.: US 2008/0091234 A1
(43) Pub. Date: Apr. 17, 2008

(54) Method for modifying a medical implant surface for promoting tissue growth

(76) Inventor: Stephanie M. Kladakis, Stoneham, MA (US)

Correspondence Address: Kirkpatrick & Lockhart Preston Gates Ellis LLP;(FORMERLY KIRKPATRICK & LOCKHART NICHOLSON GRAHAM), STATE STREET FINANCIAL CENTER, One Lincoln Street, BOSTON, MA, 02111-2950, US

(21) Appl. No.: 11/904,482

(22) Filed: Sep. 26, 2007

Related U.S. Application Data
(60) Provisional application No. 60/847,310, filed on Sep. 26, 2006.

(51) Int. Cl. A61B 17/03; B29C 59/00

(52) U.S. Cl. 606/213; 264/129

(57) ABSTRACT

Disclosed is an occluder for closing an intracardiac defect, such as a patent foramen ovale (PFO), and a method for making the same. The occluder includes a frame and at least one scaffold which are formed from a bioabsorbable polymer, such as poly-4-hydroxybutyrate. The surface of the frame and scaffold are textured to promote cell attachment. Texturing of the surface can be achieved by any number of mechanical or chemical procedures. The device is coated with collagen and heparin which are covalently bound to the surface of the device. The occluder provides improved defect closure compared to other septal occluders known in the art. In particular, the occluder described is specifically designed to improve host cell attachment to and tissue ingrowth over the device when implanted in a patient as compared to the level of host cell attachment and tissue ingrowth achieved with other implantable devices made of bioabsorbable polymers.

### CROSS-REFERENCE TO RELATED APPLICATIONS

This application claims priority to and the benefit of U.S. Provisional Patent Application No. 60/847,310, filed Sep. 26, 2006, the contents of which are incorporated by reference herein.

### BACKGROUND OF THE INVENTION

A patent foramen ovale (PFO) is a persistent, one-way, usually flap-like opening in the wall between the right atrium and left atrium of the heart. Since left atrial (LA) pressure is normally higher than right atrial (RA) pressure, the flap typically stays closed. Under certain conditions, however, RA pressure can exceed LA pressure, creating the possibility for right to left shunting of blood, permitting blood clots to enter the systemic circulation. In utero, the foramen ovale serves as a physiologic conduit for right-to-left shunting. After birth, with the establishment of pulmonary circulation, the increased left atrial blood flow and pressure results in functional closure of the foramen ovale. This functional closure is subsequently followed by anatomical closure of the two overlapping layers of tissue: the septum primum and septum secundum. However, autopsy studies have shown that a probe-detected patent foramen ovale (PFO) persists in up to approximately 25% of adults. Using contrast echocardiography (TEE), a patent foramen ovale can also be detected in approximately 25% of adults.

Studies have confirmed a strong association between the presence of a PFO and the risk for paradoxical embolism or stroke. Although the cause of ischemic stroke is not known, in approximately 40% of cases paradoxical embolism via a PFO is considered in the diagnosis, especially in young patients. In addition, there is evidence that patients with PFO and paradoxical embolism are at increased risk for future, recurrent cerebrovascular events.

Although the presence of a PFO has no therapeutic consequence in an otherwise healthy adult, patients suffering a stroke or transient ischemic attack (TIA) in the presence of a PFO and without another identifiable cause of the ischemic stroke are considered for prophylactic therapy to reduce the risk of a recurrent embolic event. These patients are commonly treated with oral anticoagulants, which have potential adverse side effects, such as hemorrhaging, hematoma, and interactions with a variety of other drugs. In certain cases, such as when anticoagulation is contraindicated, surgery may be used to close a PFO. Suturing a PFO closed typically requires attachment of the septum secundum to the septum primum with either continuous or interrupted sutures under direct visualization for example, by a thoracotomoy, or via port access surgery.

Nonsurgical closure of PFOs has become possible with the advent of implantable umbrella closure devices and a variety of other similar mechanical closure designs, developed initially for percutaneous closure of atrial septal defects (ASD). These devices allow patients to avoid the potential side effects often associated with anticoagulation therapies. However, currently available designs of septal closure devices present drawbacks, such as technically complex implantation procedures, high complication rates (for example, thrombi, device fractures, conduction system disturbances, perforations, and residual leaks), a high septal profile, and presentation of large masses of foreign material. In addition, since many septal closure devices were originally designed to close ASDs, which are true holes, rather than the flap-like anatomy of most PFOs, many closure devices lack the anatomic conformability to effectively close a PFO. In addition, some septal closure devices are complex to manufacture, which can result in lack of consistency in product performance.

A need exists for a septal closure device or occluder that can provide complete closure of a PFO in a minimum amount of time, that has a lower complication rate, and that is simple and inexpensive to use and manufacture.

### SUMMARY OF THE INVENTION

The invention is directed to septal occluders and methods of manufacturing the same. Occluders according to the invention have a frame and scaffold surface engineered to encourage cardiac tissue growth, such that the patient's own cells (host cells) completely cover the implant and close a cardiac defect, such as a patent foramen ovale (PFO). Accordingly, the invention discloses methods to enhance host cell attachment to and tissue growth over a septal occluder, although such methods can be used with any implanted medical device such as, but not limited to, a device made of bioabsorbable material.

The invention describes configuring the surface of the septal occluder so that host tissue grows over the device, healing the patient's defect without excessive fibrosis or elevated risk of thrombosis.

According to one aspect, the invention is a device for closing an intracardiac defect, such as a patent foramen ovale. For example, in one embodiment, the device includes a frame supporting at least one scaffold. The frame and the scaffold are formed of a bioabsorbable polymer. The surface of the scaffold and/or frame is textured to promote cell attachment and is coated with collagen and heparin. The collagen and heparin are covalently bound to the surface of the scaffold and/or frame. In one embodiment, the collagen is Type I collagen, while in another embodiment, the collagen is Type III collagen. Alternatively, the collagen may be recombinant human Type I or Type III collagen.

In another embodiment, the surface of the scaffold and/or frame formed from a bioabsorbable polymer is plasma treated with O₂ or with N₂ or with amine gas, while in yet another embodiment, the bioabsorbable polymer is plasma treated with amine gas and O₂.

In yet another embodiment, the bioabsorbable polymer is poly-4-hydroxybutyrate. In a further embodiment, the scaffold and frame are formed only of polymers. For example, in one embodiment, the scaffold and frame are formed from only poly-4-hydroxybutyrate, while in another embodiment, the scaffold and frame are formed from only a blend of polymers, while in a further embodiment, the scaffold and frame are formed from only one polymer.

In another aspect, the invention is a method of manufacturing an occlusion device for closing an intracardiac defect. The method includes the steps of forming a septal occluder from a scaffold and frame comprising a bioabsorbable polymer, texturing the surface of the scaffold and/or frame, and covalently binding collagen and heparin to the surface of the scaffold and/or frame. In one embodiment, the polymeric scaffold and/or frame are formed prior to being coated with collagen and heparin. In a further embodiment, collagen is coated on the pre-formed polymeric scaffold and/or frame in a step separate from coating the scaffold and/or frame with heparin. In a further embodiment, other than coating the scaffold and frame formed from a bioabsorbable polymer with collagen and heparin, no other polymer is coated on the scaffold and frame.

The bioabsorbable polymer can be textured according to a variety of methods. For example, in one embodiment, the surface of the scaffold and/or frame is textured by mechanical roughening, while in another embodiment, the surface of the scaffold and/or frame is textured by extrusion and puncturing. In another embodiment, the surface of the scaffold and/or frame is textured during the formation process by casting the polymer in a mold with a roughened surface, for example, by injection molding.

In a further embodiment, the surface of the scaffold and/or frame made of a bioabsorbable polymer can be plasma treated. For example, in one embodiment, the surface of the scaffold and/or frame is plasma treated with amine gas. In another embodiment, the polymer is treated with both amine gas and O₂.

According to another aspect, the invention is a method of closing an intracardiac defect. The method includes the steps of implanting an intracardiac occluder at the site of an intracardiac defect, for example, a patent foramen ovale, in a patient. The implanted intracardiac occluder has a frame supporting at least one scaffold. The frame and the scaffold are formed of a bioabsorbable polymer. The bioabsorbable polymer is textured to promote cell attachment and is coated with collagen and heparin. The collagen and heparin are covalently bound to the bioabsorbable polymer.

### BRIEF DESCRIPTION OF THE FIGURES

FIG. 1 is a bar graph showing the effects of plasma treatment on proliferation of HAEC cells (human aortic endothelial cells) as a function of DNA concentration on untreated polyester scaffold typically used in a septal occluder (“Polyester”), untreated bioabsorbable polymer scaffold (“Untreated”), and bioabsorbable polymer scaffold that was plasma-treated with ionized gases (“O₂”, oxygen; “N₂”, nitrogen; “NH₃”, amine). Standard tissue culture plastic was used as a control (“TCP”).

FIG. 2 is a plot of contact angle (in degrees) over time (weeks) for plasma treatment of the surface of a septal occluder with N₂, O₂, and NH₃ ionized gas, relative to controls.

FIG. 3A shows molecular weight data collected at day 4 and 5 weeks after plasma treatment of P4HB scaffold material with various gases. FIG. 3B depicts the data from FIG. 3A in bar graph format.

FIG. 4 is a bar graph of HAEC cell proliferation (as a function of DNA concentration) on a bioabsorbable polymer occluder scaffold coated with collagen type I (“Collagen I”) or collagen type III (“Collagen III”), porcine small intestinal collagen material (“ICL”), untreated bioabsorbable polymer scaffold (“Untreated”), and untreated polyester scaffold typically used in a septal occluder (“Polyester”).

FIGS. 5A-5C are a set of three photographs showing an uncoated septal occluder of bioabsorbable polymer scaffold (FIG. 5A), a septal occluder of bioabsorbable polymer scaffold coated with ICL (FIG. 5B), and a septal occluder of bioabsorbable polymer scaffold, coated with collagen type I (FIG. 5C), as implanted in a sheep.

FIGS. 6A-6F are a set of six micrographs. FIGS. 6A, 6B and 6C show an uncoated occluder frame (FIG. 6A), and the frame coated with covalent bovine collagen I (FIG. 6B), and the frame coated with covalent bovine collagen I after a durability test (FIG. 6C). FIGS. 6D, 6E and 6F show an uncoated bioabsorbable polymer scaffold (FIG. 6D), a bioabsorbable polymer scaffold coated with covalent bovine collagen I before a durability test (FIG. 6E), and a bioabsorbable polymer scaffold coated with covalent bovine collagen I after a durability test (FIG. 6F).

FIG. 7 is a bar graph representing DNA concentration in ng/mL measured at days 1, 4, and 7 on scaffold samples initially seeded with cells on day 0. The DNA concentration is an indication of the level of cell proliferation and tissue growth on the scaffold.

FIGS. 8A-8C are a set of three photomicrographs showing the unroughened surface of a septal occluder frame (FIG. 8A), the mechanically roughened surface (FIG. 8B), and the surface character of a cast bioabsorbable polymer film material (FIG. 8C).

FIG. 9 is a bar graph representing DNA concentration in ng/mL measured at days 1, 4, 7 on scaffold samples initially seeded with cells on day 0. The DNA concentration is an indication of the level of cell proliferation and tissue growth on the scaffold.

FIG. 10 is a bar graph representing the % thrombus generation for various treated P4HB surfaces (Groups I, III, and IV) as compared to P4HB treated with collagen alone (Group II).

### DETAILED DESCRIPTION

In general, a typical septal occluder includes a frame with scaffold material attached to the frame. The frame apposes the cardiac septum and provides support to the scaffold material, closing an intracardiac defect, for example, a patent foramen ovale (PFO). The scaffold material both covers the defect and provides surface area for host cell migration and attachment to and tissue growth at the site of the defect, thereby encouraging anatomical closure of the defect.

According to the invention, the closure of a patent foramen ovale can be improved by modifying the surface of the scaffold and/or frame of a septal occluder to minimize device-induced thrombosis while accelerating formation of granulation tissue and re-endothelialization (i.e., healing and cell migration and tissue growth) at the site of defect.

In one embodiment, the frame can be formed of any biocompatible metal or polymer, bioabsorbable polymer, or a shape memory polymer. In another embodiment, the tissue scaffold can be formed of any flexible, biocompatible material capable of promoting host tissue growth including, but not limited to, polyester fabrics, Teflon-based materials, such as ePTFE, polyurethanes, metallic materials, polyvinyl alcohol (PVA), extracellular matrix (ECM) or other bioengineered materials, bioabsorbable polymers, or other natural materials (e.g., collagen), or combinations of these materials. Furthermore, the surface of the tissue scaffold can be modified with drugs or biological agents to improve defect healing and/or to prevent blood clotting.

The scaffold can be attached to a septal occluder frame or to another scaffold by sutures, heat treatment, adhesives, or any other chemical bonding process.

Exemplary bioabsorbable polymers for use in making septal occluder frames and/or scaffolds include polyhydroxyalkanoates, for example poly-4-hydroxybutyrate (P4HB). Such materials are strong and flexible, but also bioabsorbable. Accordingly, it is necessary to ensure that sufficient host tissue ingrowth to close the defect occurs at the implantation site prior to complete absorption of the device. However, given that materials such as P4HB have a surface charge that discourages cell adherence to and tissue growth on the material, Applicants have developed methods for treating the surface of P4HB in order to overcome this and other barriers to cell adherence and tissue growth inherent in the material.

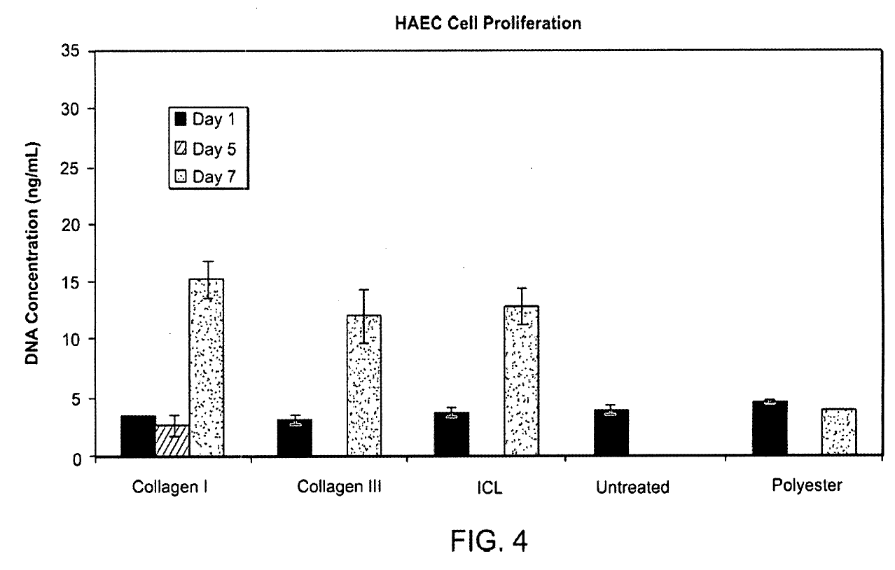

The methods employed by Applicants encourage cell attachment and tissue growth on the surface of P4HB, whereas prior to Applicants' discoveries, facilitating cell attachment and tissue growth on P4HB was problematic due to its surface properties. For example, as shown in FIG. 4, discussed in greater detail below, untreated P4HB does not facilitate cell proliferation. Given the challenges of facilitating cell attachment and tissue growth on materials such as P4HB, one aspect of the invention discloses methods for modifying devices made of bioabsorbable materials, such as P4HB, in order to promote enhanced cell attachment and host tissue growth at the site of a defect prior to absorption of the septal occluder or other implanted device made of such bioabsorbable material by the host tissue

Methods for treating P4HB as disclosed herein are applicable to other bioabsorbable materials, including other bioabsorbable polymers and can be used to improve cell attachment to and encapsulation by tissue growth of any implantable device made of a bioabsorbable polymer, such as P4HB.

In selecting a surface treatment to induce the patient's (host's) own tissue growth over the device after implantation, preference may be given to agents and methods already approved for human use. For example, in the case of bioabsorbable materials, preference may be given to a bioabsorbable material already approved for use in humans. According to one embodiment of the invention, the bioabsorbable material is given a surface treatment to promote tissue growth at the site of a defect, effectively closing the defect before absorption of the device by the host. For example, the surface treatment encourages host tissue cells to proliferate, migrate and attach to the occluder at a faster rate than if the surface were untreated, thereby closing the cardiac defect.

According to one embodiment, the surface of the device can be modified, for example, by plasma treatment. Plasma is a partially ionized gas that is generated by applying an electrical field to the gas (such as, but not limited to, O₂ gas, N₂ gas, or a nitrogen-containing gas (e.g., amine, amide, nitrile, etc.)) under at least a partial vacuum. A combination of gases may also be used. Plasma treatment changes the polarity of the material's surface, thereby increasing the surface wetability of the device, and improving the attachment of cells to the material.

According to another embodiment of the invention, cell attachment is improved by roughening or texturing the surface of the device. For example, modification of the surface morphology can promote attachment of cells or blood components to the device (Frazier, O. H. et al. (1993), “Immunochemical identification of human endothelial cells on the lining of a ventricular assist device,” Tex. Heart Inst. J. 20(2):78-82).

Roughening or texturing the surface can be achieved by either mechanical or chemical means. For instance, in one embodiment, the surface of, for example, a bioabsorbable polymeric scaffold or frame, formed from a material such as P4HB is roughened with sandpaper, sandblasted, clamped between two files, or rolled between two files. In yet another embodiment, the surface of the scaffold and/or frame is wrapped with a porous film, such as film made of a polymer such as P4HB which is then bonded to the surface by heat treatment, adhesives, or ultrasonic energy. The process of bonding causes bubbling. These bubbles create divots and bumps on the surface, thereby creating a textured or roughened surface that promotes cell attachment.

Alternately, the surface of a device such as a frame or scaffold made from a polymer can be textured or roughened by the process for forming the device. For example, in one embodiment, a bioabsorbable polymer is placed in a mold having bumps and/or divots to form a device having a roughened surface. In an alternate embodiment, the scaffold or frame is formed from a bioabsorbable polymer by a solvent casting method that generates a textured surface through formation of bubbles that form bumps or divots on the surface once the solvent evaporates. Solvent casting methods are well known in the art. In another embodiment, a polymer is melt blown to create a texture. Melt blowing produces fibrous webs or articles directly from polymers or resins using high-velocity air or another appropriate force to attenuate the filaments. Alternatively, according to another embodiment, a polymer may be extruded and then punched with a device to create holes in the polymer, thereby creating a roughened surface texture.

In a particular embodiment, the surface of the frame is textured by forming the frame in a mold that creates a textured surface, or by wrapping the frame with a porous film, such as a film of a bioabsorbable polymer as described above, while the scaffold is textured by either a melt-blowing process, an extrusion process, or a solvent casting process.

According to another embodiment of the invention, the surface of the device can be coated with or bonded to a substance that encourages cell attachment and tissue growth. For instance, in one embodiment, the scaffold and/or frame is coated with collagen, for example Type I or Type II collagen. Collagen can be coated on a scaffold by a dip process as described below. Alternately, collagen can be covalently bound to the scaffold, for example, by using UV light.

In another embodiment, extracellular matrix (ECM) is coated onto the surface of a septal occluder in order to increase cell attachment. In another embodiment, human serum increases cell attachment (Jarrell, B. E. et al. (1991), “Optimization of human endothelial cell attachment to vascular graft polymers,” J. Biomech. Eng. 113(2): 120-2). Fibronectin, larninin, and vitronectin are also promising molecules for improving cell attachment (Walluscheck, K. P. et al. (1996), “Improved endothelial cell attachment on ePTFE vascular grafts pretreated with synthetic RGD-containing peptides,” Eur. J. Vasc. Endovasc. Surg. 12(3):321-30; Wigod, M. D. and B. Klitzman (1993), “Quantification of in vitro endothelial cell attachment to vascular graft material,” J. Biomed. Mater. Res. 27(8):1057-62). Fibronectin can be bound to the surface of a device such as a septal occluder with TDMAC (trododecylmethylammonium chloride), a cationic surfactant. Most of the above-identified compounds can be coated on a device at a concentration of about 40 micrograms/ml.

In another embodiment, peptides and other biological molecules that serve as chemoattractants can be used to coat the devices, where the chemoattractants attract and retain the cells. For example, RGD and REDV are peptides of three and four residues, respectively, which can be bound to ePTFE via poly-L-lysine and glutaraldehyde, or crosslinked to peptide fluorosurfactant polymer (PFSP) and adsorbed onto ePTFE (Walluscheck, K. P. et al. (1996), “Improved endothelial cell attachment on ePTFE vascular grafts pretreated with synthetic RGD-containing peptides,” Eur. J. Vasc. Endovasc. Surg. 12(3):321-30; Larsen, C. C. et al. (2006), “The effect of RGD fluorosurfactant polymer modification of ePTFE on endothelial cell attachment, growth, and function,” Biomaterials 27(28):4846-55). These polypeptides bind to cell surface receptors, thereby adhering cells to the surface of the material.

In another embodiment, molecules such as MCP-1, VEGF, FGF-2 and TGF-beta are applied to a septal occluder in order to stimulate wound repair (e.g., angiogenesis and formation of granulation tissue) at the site of the defect, thereby attracting host cells to the defect. Applied to a septal occluder, or combined with a tissue repair fabric, e.g., ICL (intestinal collagen layer (Organogenesis, Inc., Canton, Mass., USA), these bioactive components may promote endothelial cell migration and proliferation and accelerate healing of a PFO. Gels (e.g., REGRANEX (Ethicon Inc., Somerville, N.J., USA)) containing recombinant human growth factors may be added either singly or in combinations to ICL, for example, on a septal occluder.

In another embodiment, antibodies to cell surface markers can be used to coat the devices. Antibodies can be designed to attract and retain a cell type with greater specificity than collagen. Such antibodies would be designed to interact with a specific cell surface antigen of the target cell type.

In another embodiment, the scaffold and/or frame is coated with molecules having a charge opposite to molecules occurring on the host's target cells. This causes these cells to bond with the occluder surface, allowing cell attachment and host tissue growth to occur at the site of the defect.

These various methods can be combined to achieve better cell attachment and tissue growth on an implanted intracardiac device. For instance, an implant can be surface-textured by wrapping in a porous film which is then thermally bonded to the implant, and then dip-coating the wrapped implant in collagen or ECM components.

In a further embodiment, heparin is coated on the scaffold and/or frame of the device to reduce the occurrence of thrombogenic events, such as blood clotting, at the site of implantation. Heparin, in one embodiment, is covalently linked to the scaffold by exposure to UV light. In another embodiment, heparin is coated on the scaffold by a dipping process where the scaffold is dipped in, for example, a solution of heparin benzalkonium chloride (H-BAC) (North American Science Associates, Inc, Northwood, Ohio) and the heparin-coated scaffold is then dried.

In another embodiment, the surface of a scaffold and/or frame is treated with two or more treatment types to encourage cell attachment and tissue growth. For example, in one embodiment, the surface is textured according to any of the methods previously discussed and the surface is plasma treated as previously discussed. In yet another embodiment, the surface is textured and coated with collagen and/or heparin. In a further embodiment, the surface is textured and plasma treated. Alternately, the surface is textured, plasma treated, and coated with collagen and/or heparin.

According to another embodiment, both collagen and heparin are coated on the polymer scaffold and frame of the intracardiac occluder. In one embodiment, the polymeric scaffold and/or frame are formed from a bioabsorbable polymer then next, the frame and scaffold are coated with collagen and heparin. In a further embodiment, collagen is coated on the pre-formed polymeric scaffold and/or frame in a step separate from coating the frame with heparin. In a further embodiment, other than coating the polymeric scaffold and frame with collagen, no other polymer is coated on the scaffold and frame.

### EXAMPLE 1

### Surface Modification Through Plasma Treatment

Plasma treating the surface of a septal occluder is one way to alter the surface characteristics of the material to promote protein deposition and cell attachment. Plasma treating the septal occluder increases the wetability of the implant surface, thereby improving endothelial cell attachment to the implant.

Plasma is partially ionized gas generated by applying an electrical field to a gas under at least partial vacuum. Plasma reacts and combines with first few atomic layers of the surface while the visual and bulk properties of the material remain unchanged. Gases such as oxygen and nitrogen have been used during plasma treatment, as well as gases containing amine groups.

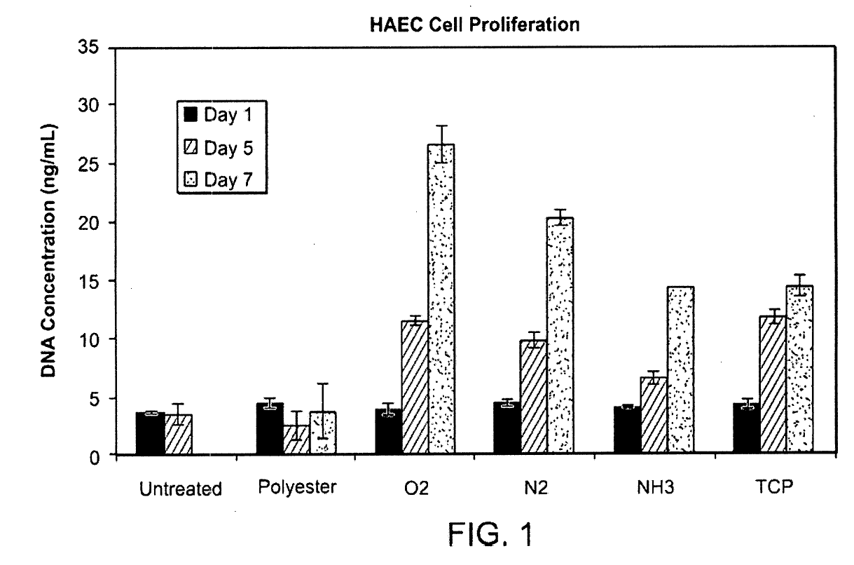

FIG. 1 is a bar graph showing the effects in vitro of plasma treatment on proliferation of HAEC cells (human aortic endothelial cells) on an untreated polyester scaffold typically used in a septal occluder (“Polyester”), an untreated bioabsorbable polymer scaffold (P4HB) (“Untreated”), and a bioabsorbable polymer scaffold (P4HB) that was plasma-treated with ionized gases (“O₂”, oxygen; “N₂”, nitrogen; “NH₃”, amine). Standard tissue culture plastic was used as a control (“TCP”).

The results show that plasma treatment improved cell attachment to the bioabsorbable polymer scaffold (P4HB) over the untreated bioabsorbable polymer scaffold, especially when the ionized gas used was oxygen or nitrogen.

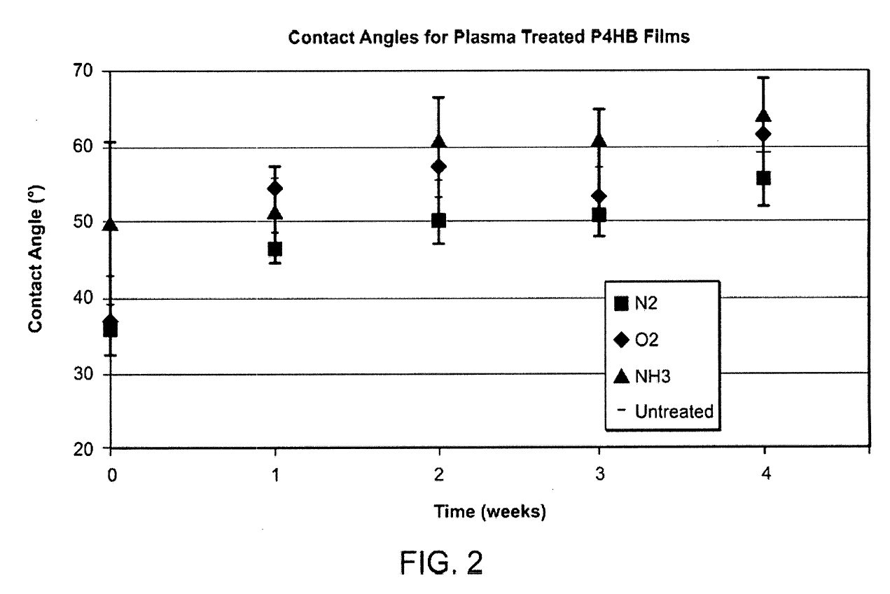

Plasma treatment of the P4HB appears to relatively stable. FIG. 2, shows a graph of contact angle (in degrees) over time (weeks) for P4HB plasma treated with N₂ (squares), O₂ (diamonds) and NH₃ (triangles) ionized gas, relative to controls (−). Plasma treatment reduces the contact angle of the treated surface and therefore improves its wetability. As shown in FIG. 2, the contact angle after plasma treatment increases slightly and levels off over time. This means that wetability of the treated surface will decrease slightly within a short period of time after plasma treatment, and eventually will be maintained at a constant level. Since wetability of a surface is directly related to its ability to allow cells to attach to it, the data indicates that while improved cell proliferation achieved by plasma treatment may decrease slightly within a short period of time after the plasma treatment, cell proliferation should remain relatively stable thereafter.

### EXAMPLE 2

### Stability of Plasma Treated Bioabsorbable Polymers

In order to determine the stability of plasma treated P4HB, molecular weight data of plasma treated solvent cast (porous cast) P4HB samples was taken at 4 days after plasma treatment and 5 weeks after plasma treatment.

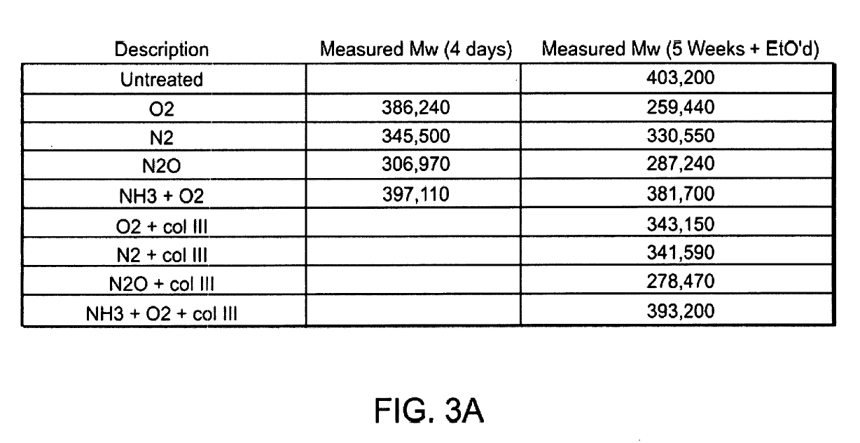

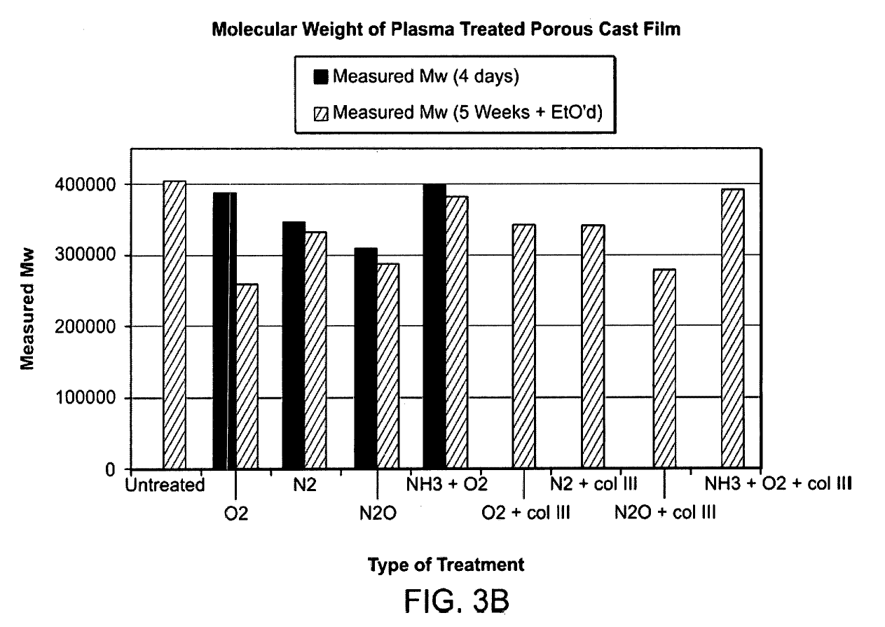

Plasma treated samples were processed at PLASMAtech (Erlanger, Ky.). Samples of P4HB were plasma treated with oxygen gas (O₂), nitrogen gas (N₂), nitrous oxide (N₂O), and a combination of ammonia gas (NH₃) and oxygen gas (O₂). The molecular weight data, shown in FIGS. 3A-B, indicates that the decrease in molecular weight on a percentage basis was least for the combination NH₃/O₂ treated P4HB. Accordingly, the NH₃/O₂ plasma treated P4HB has the greatest stability of the plasma treatments tested. According to the invention, the ratio of NH₃ to O₂ used to treat the P4HB is 2:3 in one embodiment, 1:1 in another embodiment, and 1:2 in yet another embodiment.

### EXAMPLE 3

### Surface Modification by Collagen Coating

Collagen can be made recombinantly in highly purified form, free of contamination from disease-causing pathogens such as viruses and prions. It is also available commercially (e.g., from FibroGen, Inc., South San Francisco, Calif., USA). Collagen type I and type III promote tissue growth, and can be applied to the surface of a medical implant through a simple dip coating process. For example, to coat a P4HB scaffold or frame with collagen according to the invention a 40 microgram/mL solution of collagen is made by combining 0.5 mL liquid collagen with 37.5 mL PBS. The scaffold or frame is then cleaned with ethyl alcohol and deionized water prior to being soaked in the collagen solution for 15 minutes. The scaffold or frame is dried for one hour between coats. Any number of coats of collagen may be applied. Four (4) coats of collagen are optimal according to one embodiment.

Attachment of HAEC cells to variously-treated scaffolds is shown in FIG. 4, which is a bar graph of HAEC cell proliferation (as a function of DNA concentration) on a bioabsorbable occluder scaffold (P4HB) coated with collagen type I (“Collagen I”), a bioabsorbable occluder scaffold (P4HB) coated with collagen type III (“Collagen III”), a scaffold of porcine small intestinal collagen material (“ICL”), a scaffold of untreated bioabsorbable polymer scaffold (P4HB) (“Untreated”), and an untreated polyester scaffold typically used in a septal occluder (“Polyester”).

FIG. 4 shows that coating the scaffold with collagen I or collagen III improves attachment of HAEC cells to an extent similar to the level of cell attachment seen with ICL. In addition, FIG. 4 shows that by day 7, the collagen treated scaffold provides significantly greater levels of cell proliferation than the untreated P4HB scaffold or the polyester scaffold. In fact, by day 7 no cells were growing on the untreated scaffold, indicative that the untreated material is not conducive to tissue growth.

These results were confirmed in vivo, in a sheep model. In this model, atrial defects were created in the animal using the Brockenbrough technique, which involved puncturing the atrial septum with a needle from the right atrium to gain access to the left atrium. A balloon catheter was then inserted into the puncture site, passed across the atrial septum, and inflated to the desired diameter to create a defect in the atrial septum. The implant was then percutaneously deployed at the defect site.

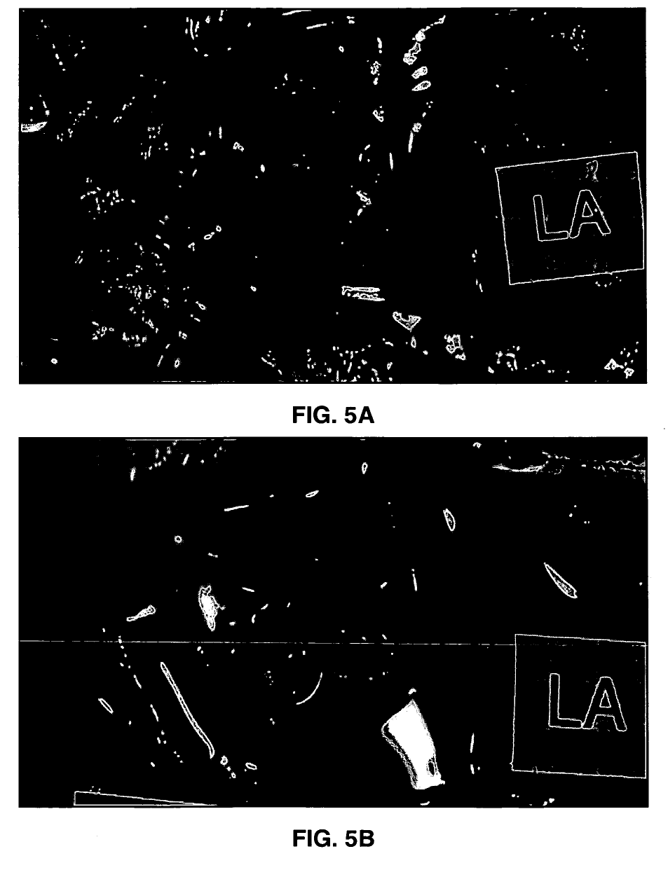

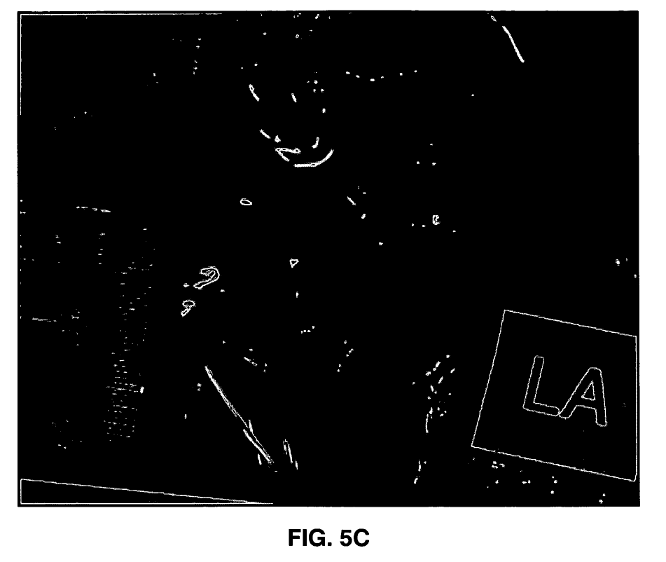

The set of three photographs in FIG. 5 shows the amount of tissue coverage after one month on an uncoated septal occluder of bioabsorbable polymer scaffold (P4HB) (FIG. 5A), a septal occluder of ICL (FIG. 5B), and a septal occluder of bioabsorbable polymer scaffold (P4HB), coated with collagen type I (FIG. 5C).

These results are also shown quantitatively in Table 1, below. “Sealing” represents the amount of attachment between the circumference of the scaffold to the intracardiac septum. “Encapsulation” represents amount of tissue coverage to the scaffold. These results show that bioabsorbable polymer scaffold (P4HB) coated with collagen Type I performs similarly to the ICL scaffold.

TABLE 1
Sealing and Encapsulation of the Septum by Treated Scaffolds

| | Collagen Type I | | Uncoated |
|---|---|---|---|
| | coated bioabsorbable | ICL | Bioabsorbable |
| | polymer scaffold | Scaffold | Polymer scaffold |
| Encapsulation to | 1.0 | 1.4 | 0.6 |
| Sealing Ratio | | | |
| Encapsulation | 50-75% | 25-50% | <25% |
| Sealing | 50-75% | <25% | 50-75% |

### EXAMPLE 4

### Covalently Bound Extracellular Matrix Components (ECM)

Extracellular Matrix (ECM) components are naturally occurring molecules that are found in the matrix surrounding cells. In this study, the effects of ECM coatings on the durability of occluder frames and bioabsorbable polymer scaffold (P4HB) were determined. Collagen I was covalenty coated onto the devices by a dip process with ultraviolet (UV) exposure. Durability was tested by simulating the deployment of the device six times in an aqueous environment at 37° C.

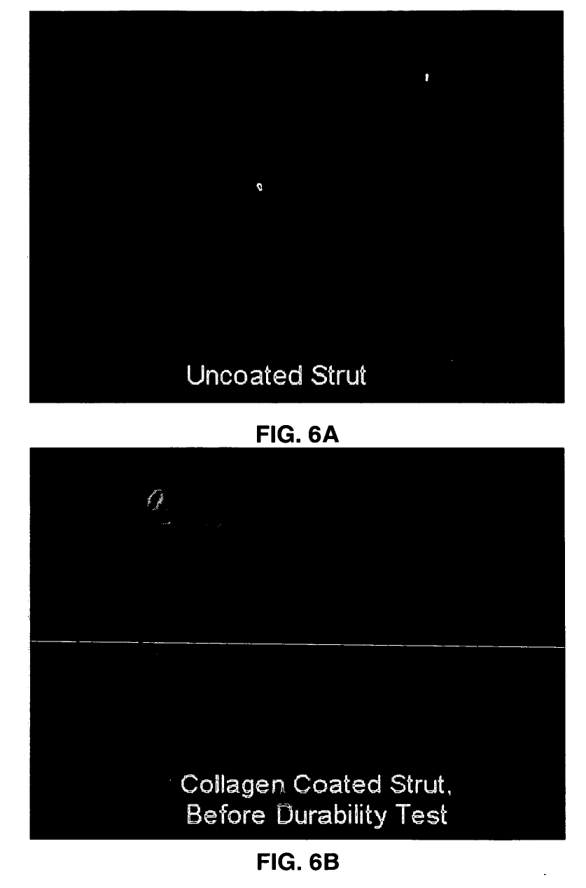

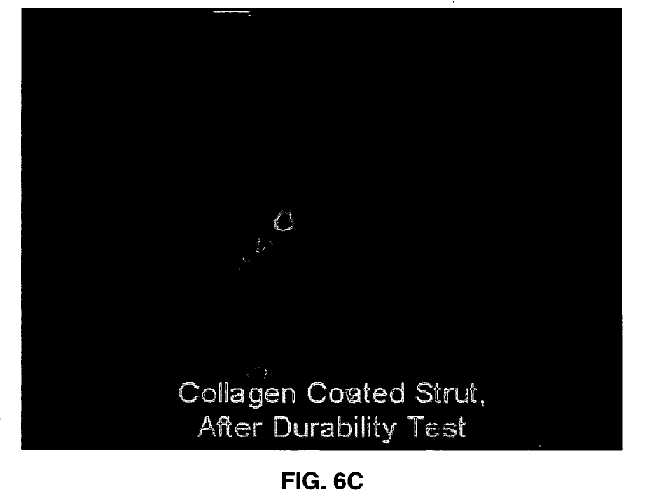

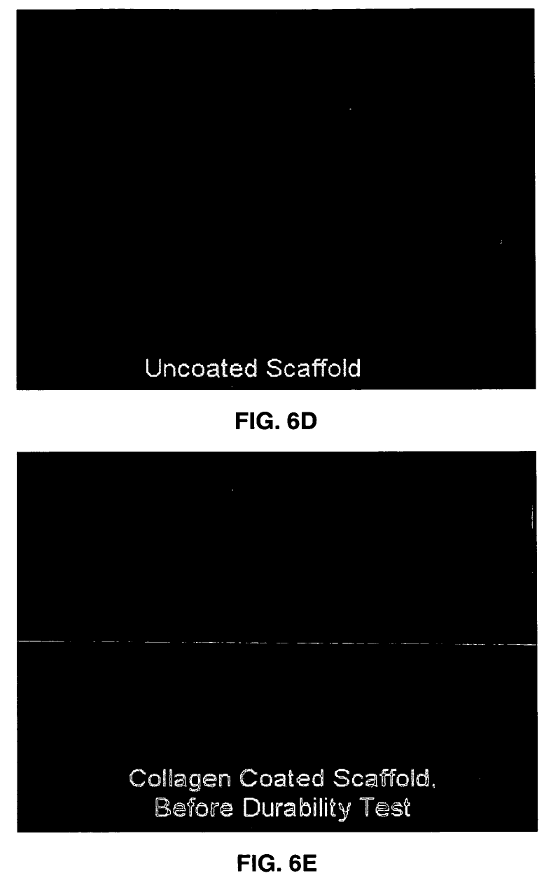

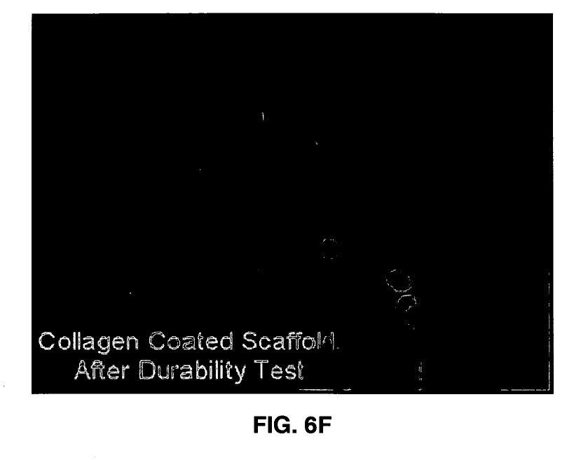

The set of six micrographs if FIG. 6 shows the results of this study. FIGS. 6A, 6B and 6C show an uncoated occluder frame (FIG. 6A), the frame coated with covalent bovine collagen I (FIG. 6B), and the frame coated with covalent bovine collagen I after a durability test (FIG. 6C). FIGS. 6D, 6E and 6F show an uncoated bioabsorbable polymer scaffold (FIG. 6D), a bioabsorbable polymer scaffold coated with covalent bovine collagen I before a durability test (FIG. 6E), and a bioabsorbable polymer scaffold coated with covalent bovine collagen I after a durability test (FIG. 6F). The dots represent human microvascular endothelial cells attached to the device. These results indicate improved cell attachment after the device is coated with covalent bovine collagen I.

### EXAMPLE 5

### Comparison of Cell Attachment and Proliferation on Plasma Treated Scaffold, Collagen Coated Scaffolds, and Combinations Thereof

In another in vitro experiment, the ability of various treatments to scaffold material were compared in their ability to facilitate HAEC cell proliferation were compared. Scaffold material was cut into ⅝ inch diameter discs. Pieces of P4HB scaffold material were sent to PLASMAtech (Erlanger, Ky.) for plasma treatment with O₂ (Group A), N₂ (Group C), N₂O (Group E), and a combination of NH₃ and O₂ (Group G). In addition, a sample of each of the plasma treated groups was also subjected to coating with collagen III by a dip process as previously described (O₂ and Collagen III (Group B), N₂ and Collagen III (Group D), N₂O and Collagen III (Group F), and a combination of NH₃ and O₂ and Collagen III (Group H). Further, samples of P4HB were subjected to coating in Collagen I only by a dip process (Group I), coating in only Collagen III by a dip process (Group K), or coating in Collagen I by a covalent binding process involving UV exposure. Untreated P4HB (Group M) and heparin benzalkonium chloride (H-BAC) coated ICL material (Group N) were also tested. Tissue culture plastic was used as a control (Group O).

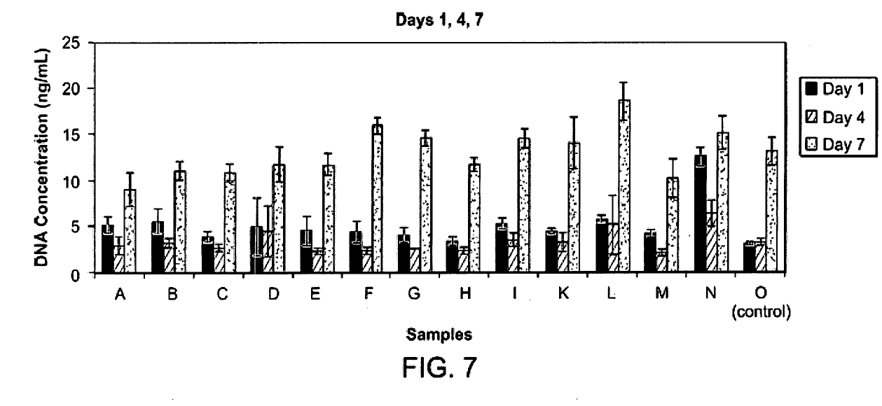

Scaffold were conditioned in a medium for 24 hours prior to cell seeding after which scaffolds were seeded at a density of 10,000 cells per scaffold. Cell proliferation was evaluated at 3 points after seeding at day 1, day 4, and day 7 via a DNA concentration assay (Quant-iT, Pico Green dsDNA Assay). Results are shown in FIG. 7.

A comparison between the untreated porous cast film (Group M) and the plasma treated porous cast film (Groups A-H) reveals that at the 7 day time point, the DNA concentrations of plasma treated groups F and G had a significantly higher DNA concentration than the untreated porous cast film (Group M). Further, comparison of cell proliferation on the collagen treated samples (Groups I-L) showed that scaffold treated with covalently bound collagen (Group L) experienced better cell proliferation than collagen applied to the scaffold by the dipping technique described (Groups I, K).

### EXAMPLE 6

### Modification of Scaffold Surface Texture

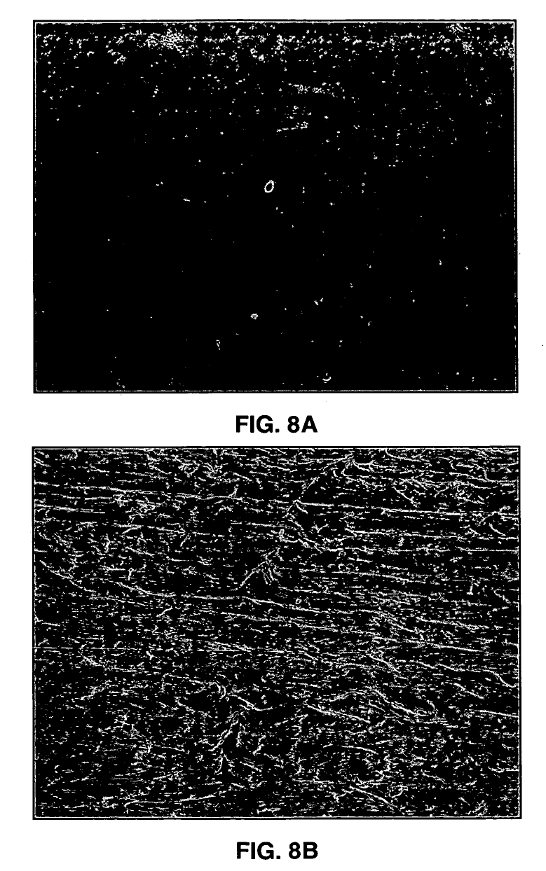

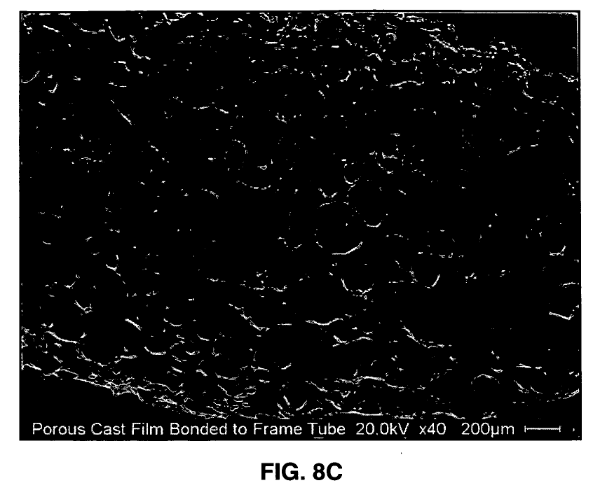

In this example, the surface of a septal occluder frame was roughened by various means to change the material surface morphology and therefore promote cell or blood component attachment. The method used was mechanical roughening accomplished by pulling the material between two sheets of 240 grit sandpaper. FIG. 8 shows photomicrographs of the unroughened surface of a septal occluder frame made of P4HB (FIG. 8A), the mechanically roughened surface of P4HB (FIG. 8B), and the surface character of a cast bioabsorbable polymer film material (P4HB) (FIG. 8C).

Other methods of altering the surface character of an implant include CO₂ particle blasting (i.e., sand blasting), etching treatment with acidic or basic solutions, wrapping with a porous film which is them thermally bonded to the surface of the implant, clamping the material between two files, or rolling it between two files. Other methods of texturing or roughening the surface have been described previously.

### EXAMPLE 7

### Heparin Coating of Scaffold Material

In order to determine the effect of the anticoagulant heparin on cell attachment and tissue growth, various combinations of surface modifications and coatings of the scaffold material were tested in vitro with or without heparin coating for comparison purposes.

Porous cast P4HB film was cut into ⅝ inch diameter circles. Some P4HB film was then plasma treated with NH₃ and O₂ gas (Group C) (PLASMAtech, Erlanger, Ky.). Some of the plasma treated film was also then further coated with H-BAC via the dipping procedure previously described (Group K). Porous cast P4HB film was also coated with covalently bound collagen I (Group B), and also further with covalently bound Heparin (Group A) or with H-BAC via the dipping procedure previously described (Group I). ICL material was also coated with H-BAC as described (Group E). Another group tested was porous cast P4HB film coated with H-BAC (Group L) and melt-blown P4HB coated with H-BAC (Group G). P4HB that had been extruded and punched with holes as described above and coated with H-BAC (Group N) was also tested. Untreated porous cast P4HB (Group D), untreated melt-blown P4HB (Group F), untreated extruded and punched P4HB (Group M) and tissue culture plastic (Group H) served as controls.

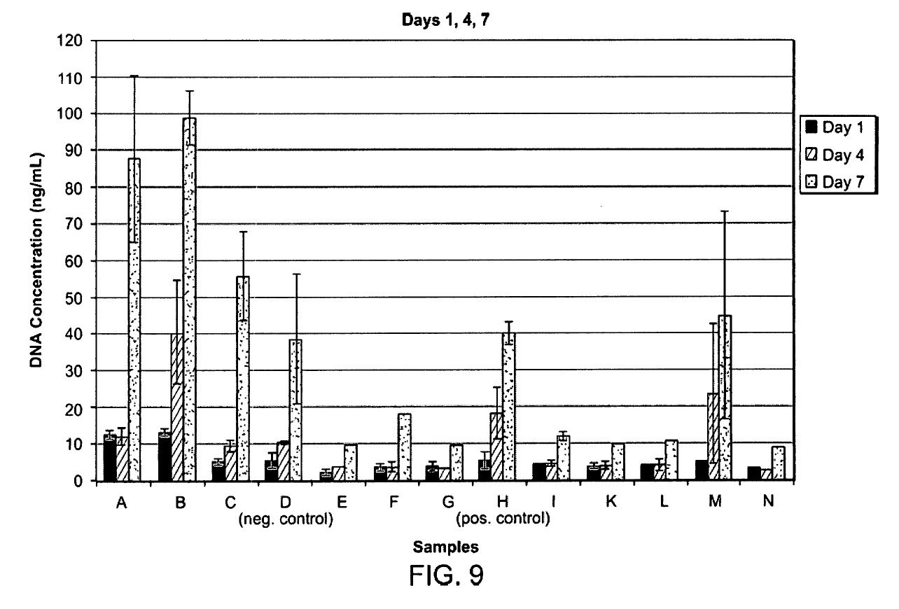

All of the above groups were conditioned in medium for 24 hours prior to cell seeding, after which they were seeded with 5000 cells/scaffold. 1 mL medium was added 1 hour after seeding and the medium was changed every 3 days. Cell proliferation was measured via the Quant-I, Pico Green dsDNA assay (Molecular Probes (Invitrogen)) at day 1, day 4, and day 7. Results are shown in FIG. 9.

As illustrated in FIG. 9, Groups A and B experienced the greatest amount of cell proliferation. Accordingly, coating the device with collagen by covalently binding the collagen to the surface of the device provides a significant improvement in cell proliferation over controls. Further, the covalently bound heparin coating (Group A) appears not to have statistically interfered with cell proliferation as compared to Group B. Accordingly, heparin coating can be used concomitantly with collagen coating without adverse affects on cell proliferation rates (i.e., tissue growth rates).

### EXAMPLE 8

### Thrombogenicity of Collagen Treated Scaffold

In order to determine if collagen coating of a bioabsorbable scaffold would have any adverse thrombogenic effects, various scaffolds were tested in an in vitro model to determine the thrombogenicity of a covalently bound collagen coated P4HB scaffold (Group 2) in comparison to a covalently bound collagen and heparin coated P4HB scaffold (Group 1), an uncoated P4HB scaffold (Group 3) and a plasma treated scaffold (Group 4).

In order to test the thrombogenicity of the variously treated scaffold materials, the devices were deployed in 25 mm ID PVC conduits. Fresh, heparinized blood (2 U/mL) was recirculated through the PVC conduits at 2.5 L/min using roller pumps for 1.5-2 hours. At the end of each experiment, the devices were photographed in situ and upon retrieval from the conduit, were placed in counting vials for measurement of radiation in a gamma counter.

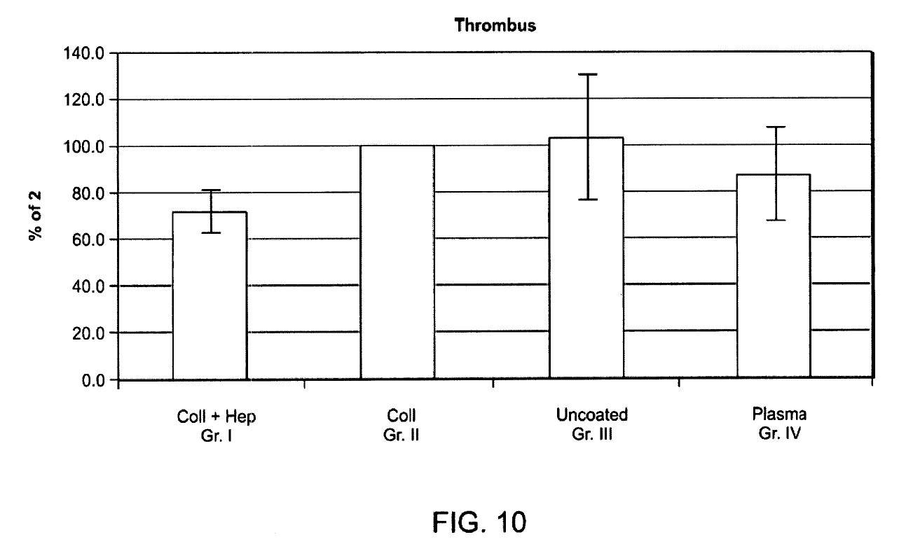

As the results in FIG. 10 show, Group 1 was associated with 35% less thrombus formation compared with Group 2. Other pair-wise differences were, however, not statistically significant. Accordingly, application of collagen does not increase the risk of thrombus formation as compared to uncoated or plasma treated P4HB; however, addition of heparin to a covalently bound collagen coated scaffold reduces thrombus formation by 35% as compared to the covalently bound collagen coating alone.

### EXAMPLE 9

### Implantation of a Bioabsorbable Occluder in a Human

A septal occluder manufactured from a bioabsorbable polymer, such as P4HB, is implanted in a human. The P4HB from which the septal occluder is formed has been textured according to any one or more of the procedures described herein. The P4HB has also been coated with collagen and/or heparin according to any one of the methods described herein.

The septal occluder according to the invention is then implanted at a cardiac defect, such as a patent foramen ovale or atrial septal defect via a percutaneous transvascular procedure using a catheter. Such implantation procedures are well known in the art. At 30 days, significant cell proliferation and tissue growth has occurred. By 90 days the occluder is completely encapsulated with host tissue such that the occluder cannot be seen. By 1 year, the bioabsorbable occluder is at least partially or completely absorbed by the host and the defect is completely closed with host tissue.

While this example is specifically focused on human implantation, such a device is contemplated for implantation in a variety of mammals such as, for example, a dog, a cat, a horse, a cow, or a pig.

1. An occluder for closing an intracardiac defect, the occluder comprising:
a frame supporting at least one scaffold, said frame and said scaffold being formed from a bioabsorbable polymer, wherein a surface of the scaffold is textured to promote cell attachment, and wherein collagen and heparin are covalently bound to the surface of the scaffold.

2. The occluder of claim 1, wherein a surface of the frame is textured to promote cell attachment, and wherein collagen and heparin are covalently bound to the surface of the frame.

3. The occluder of claim 1, wherein the surface of the scaffold is plasma treated with O₂.

4. The occluder of claim 1, wherein the surface of the scaffold is plasma treated with N₂.

5. The occluder of claim 1, wherein the surface of the scaffold is plasma treated with amine gas.

6. The occluder of claim 5, wherein the surface of the scaffold is also plasma treated with O₂.

7. The occluder of claim 1, where the collagen is Type I collagen.

8. The occluder of claim 7, where the collagen is recombinant human Type I collagen.

9. The occluder of claim 1, where the collagen is Type III collagen.

10. The occluder of claim 9, where the collagen is recombinant human Type III collagen.

11. The occluder of claim 1, wherein the bioabsorbable polymer is poly-4-hydroxybutyrate.

12. A method of manufacturing a septal occluder for closing a septal defect, the method comprising the steps of:
forming a septal occluder from a scaffold and frame comprising a bioabsorbable polymer;
texturing the surface of the scaffold; and
covalently binding collagen and heparin to the surface of the scaffold.

13. The method of claim 12, further comprising the steps of texturing the surface of the frame and covalently binding collagen and heparin to the surface of the frame.

14. The occluder of claim 12, wherein the surface of the scaffold is textured by mechanical roughening.

15. The occluder of claim 12, wherein the surface of the scaffold is textured by extrusion and puncturing.

16. The occluder of claim 12, wherein the surface of the scaffold is textured by forming the scaffold in a mold with a roughened surface.

17. The occluder of claim 12, wherein the surface of the scaffold is plasma treated with amine gas.

18. The occluder of claim 17, wherein the surface of the scaffold is also plasma treated with O₂ gas.

19. The method of claim 12, wherein the bioabsorbable polymer is poly-4-hydroxybutyrate.

20. The method of claim 12, wherein the step of covalently binding collagen to the surface of the scaffold occurs separately from the step of covalently binding heparin to the surface of the scaffold.

21. A method of closing an intracardiac defect comprising implanting the occluder of claim 1 in a patient.

22. The method of claim 21, where the intracardiac defect is a patent foramen ovale.
